(19) United States
(12) Patent Application Publication
(10) Pub. No.: US 2015/0032208 A1
(43) Pub. Date: Jan. 29, 2015

(54) DEVICES, SYSTEMS, AND METHODS FOR IMPLANT DELIVERY

(71) Applicant: KELLER MEDICAL, INC., STUART, FL (US)

(72) Inventor: Howard E. Preissman, Stuart, FL (US)

(21) Appl. No.: 14/456,952

(22) Filed: Aug. 11, 2014

Related U.S. Application Data
(63) Continuation of application No. PCT/US12/24917, filed on Feb. 13, 2012.

(51) Int. Cl. A61F 2/12

(52) U.S. Cl. CPC A61F 2/12
USPC 623/8

(57) ABSTRACT

A tapered sleeve (4) is provided for implant delivery (2). An implant (e.g., a pre-filled silicon breast implant) is introduced into a large proximal end of the sleeve and extruded into a surgical pocket of minimal access incision size through a small-sized distal end of the device. Features of the delivery system are provided to assist in sterile preparation, ensure one time use, improve the delivery of high-friction implants and/or achieve a combination of the above.

### CROSS-REFERENCE TO RELATED APPLICATIONS

This application is a continuation of PCT Application Serial No. PCT/US2012/024917, filed Feb. 13, 2012, which is incorporated herein by reference in its entirety for all purposes.

### BACKGROUND

Breast reconstruction and augmentation with pre-filled implants continues to evolve. Since the re-introduction of silicone breast implants to the US market in 2006, patient preference has driven their sales. A tapered flexible sleeve sold under the trademark, KELLER FUNNEL, has greatly improved procedural aspects of implantation of these implants.

Aspects of the KELLER FUNNEL are detailed in each of US Published Patent Application Nos.: US 2009/0204107, entitled “Apparatus and Process for Delivering a Silicone Prosthesis into a Surgical Pocket;” US 2010/0280610, entitled “Silicone Breast Implant Delivery;” and US 2011/0035003, entitled “Fail-safe Silicone Breast Implant Delivery Device,” each of which are fully incorporated by reference herein. Use of the funnel facilitates delivering implants (given their selected size) through relatively smaller incisions than possible when done by direct hand insertion. In addition, use of the funnel assists in performing the procedure in a “no touch” sterile fashion.

Many consider the instances of capsular contraction associated with breast implants to be the result of contaminant introduction during implant delivery. With the KELLER FUNNEL, hand contact can be avoided by transferring an implant from its original packaging into a prepared funnel (e.g., one soaked to activate its hydrophilic coating) such that the only contact made with the implant during implantation is done by the inner surface of the funnel itself.

While quite elegant in design, improvement in the manufacturing cost of the KELLER FUNNEL is desirable, as is improvement in its performance, especially with respect to delivery of so-called “textured” implants.

### SUMMARY

The devices, systems, and methods described herein offer new and useful improvements in the field of pre-filled, solid, and/or semi-solid implant (e.g., breast, gluteal, testicular, etc.) delivery. The implant may be described as an anatomical implant, deformable prosthesis, deformable implant and any combination thereof. The implants are typically silicone, but other implant compositions can be used as well. The devices, systems, and methods described herein are generally used with implants that are not inflated (or filled) after insertion, like most saline implants. The implant can also be a non-deformable prosthesis, such as a hard glass (or ceramic) ocular implants, pacemaker, or a cheek implant, and the like.

A truncated, generally conical sleeve is described that can be, e.g., a frustum-shaped device. The device can have two ends and be adapted to receive an implant through a larger end (measured generally by width or peripheral dimension) and squeezed to expel the implant through the smaller end into a tissue pocket. The implant delivery sleeve can be constructed of a flexible material and can be similar in size to commercially available pastry bags.

More specifically, since the size of silicone implants may vary in a range from about 150 cc to about 800 cc, the dimensions of the sleeve may vary. A proximal opening sized to fit any implant in this size range and a distal opening sized to fit the smallest implant may be desirable. The distal opening (or tip) may be enlargeable by trimming the sleeve. In fact, the device can be manufactured and sold without a distal opening, with the expectation that the user will trim the sleeve to create the appropriately sized distal opening. Indicia may be present on the exterior of sleeve as a guide for cutting to the proper dimensions. Otherwise, a template or other means may be provided to assist in trimming.

In one embodiment, the implant(s) are provided in packaged combination, or kit, with one or more delivery sleeves. In such a kit, an implant can be pre-loaded into a delivery sleeve.

As contamination is generally believed among the plastic surgeon community to have a direct effect on the incidence of infection and capsular contracture during implant surgery, there are some advantages to providing implants ready for surgical insertion in a configuration that allows them to be placed without the necessity of being handled by the surgeon or staff. It is commonly known and instructed that surgeons should put on new gloves just prior to removing sterile gel implants and inserting them into the surgical pockets previously created. By donning new gloves the risk of contamination originating for the autologous skin based flora of the patient is limited.

Another method to reduce the risk from this vector of contamination or others is to have the implants prepackaged inside a delivery sleeve eliminating the need for handling by any staff during the surgical procedure. In an embodiment where the implant is prepackaged in a KELLER FUNNEL that incorporates a dry interior coating for lubrication, a technique for hydrating this inner surface of the sleeve is also addressed.

Without touching the implant, the coating on a sleeve can be hydrated by holding the sleeve upright (proximal end up) preloaded with the implant in a manner that prevents the implant from falling out while allowing a member of the surgical staff to apply a hydrating fluid directly to the inside of the sleeve. This fluid could be one typically used in the Operating Room (OR) for such purposes currently employed for hydrating the funnel or bathing the implant. These include, but are not limited to, sterile saline, beta dyne, antibiotic, or any combination thereof. The distal end the sleeve can be dipped into a bowl of the same solution to hydrate the distal end, if needed. Hydration in combination with manual manipulation of the implant through the sleeve wall would allow adequate hydration of all interior surfaces.

While the above represents a suitable method for use with a preloaded, packaged sleeve, additional modes of packaging the sleeve may alternatively be employed to facilitate the process of complete hydration of the sleeve's coating.

Specifically, one such option involves providing an implant in a sleeve having a sealed distal end. The distal end could be temporarily sealed by way of a clip placed across the opposing sleeve surfaces, which may or may not be first folded over at the tip and then sealed with a clip. A number of clip-type devices suitable for such purposes could be employed, as will be recognized by those of ordinary skill in the art.

Alternatively, the distal end of the tapered sleeve may be a conical point, or a truncated end (e.g., in a frustum-type construction) sealed during the manufacturing process by, for example, the application of tape, the application of heat to melt the opposing surfaces together, or the heating of the dry lubricious coating itself as “hot melt” glue during the curing process. Or an alternate fluid or adhesive could be used as a glue. Any number of adhesives commonly used in the medical device industry could be used for such purposes depending on the materials used in the construction of said funnel, as will be apparent to those of ordinary skill in the art.

In either case, the extended end or manufactured seal would typically be trimmed off during the sizing process employed after hydration of the funnel. Thus, the sleeve itself acts as a receptacle for the hydrating and bathing solution, until modified for effecting implant delivery. As such, the sleeve is repurposed from a device for implant and procedure preparation into a device for implant delivery and procedure completion.

For preparation, the implant can be manually manipulated in the sleeve. And once hydrated and bathed, the clipped or sealed end can be opened, allowing the excess fluid to drain. Once the sleeve is drained, the implant can be inserted into a surgical pocket without having ever been touched by the surgeon, thereby maintaining the same or similar level of sterility as provided in the original packaging.

In connection with a pre-packaged implant, it should be recognized that it is commonly instructed that the surgeon carefully inspect the implant prior to insertion to look for visual defects. As such, the sleeve material is advantageously clear to help facilitate such inspection and do so without the need to directly handle the implant.

It is also contemplated that the sleeve may be pre-trimmed to correlate with the specific implant type and surface configuration. Alternatively, if pre-sealed, the exact location for optimum sizing of the distal end opening could be clearly marked with an indication (e.g., pad printing, silk screening, etc.) where to trim.

In yet another variation, the funnel is packaged and sold in a pre-hydrated configuration with both ends sealed containing the hydrating fluid and implant. Alternatively, the funnel could be open at both ends with the implant contained therein and packaged in such a configuration containing hydrating fluid throughout.

Regardless, for implant delivery the small end of the delivery sleeve is placed through a skin incision into a tissue pocket and pressure is applied to force the implant from the sleeve into the pocket. Such action avoids rough or gross manipulation as required by purely manual implantation. As such, use of the sleeve makes for a more “gentle” procedure reducing the potential for implant damage and/or adjacent tissue trauma. Still further, the small size of the delivery sleeve opening allows the physician flexibility in his/her surgical approach by allowing access through inframammary, periareolar, or axillary sites. In all cases, the incision is smaller and less noticeable than otherwise required for inserting silicone implants, thereby diminishing any scarring.

The devices, systems, and methods described herein have features and advantages that improve the design and use of the sleeve as compared to the originally-marketed KELLER FUNNEL. For instance, the sleeve is advantageously constructed of a smooth plastic film. It may comprise polyvinylchloride (PVC), polyethylene terephthalate (PET), or another polymer. Such materials can be produced at least substantially clear and with a substantially smooth surface.

Whether employed in a no-touch kit as described above, substantial clarity enables a user to visualize the orientation of the implant in the funnel sleeve when preparing to insert the implant into the surgical pocket. It can also allow the user to locate defects in the implant by way of inspection through the sleeve wall. The smooth surface plays a role in clarity as well, and can provide an improved base upon which to adhere a lubricous coating that retains durability.

A hyaluronic acid (HA) based hydrophilic coating applied upon a smooth film substrate used to construct the sleeve allows a significant number of implant passes to be made with no readily observable degradation in performance. One example of such is an HA-based hydrophilic coating applied by Formacoat, LLC.

This result is likely a function of the smooth sleeve as well as the difference in the coating. However, the HA coating is more expensive that other hydrophilic coatings such as the trademarked LUBRILAST coating. The HA coating on a fabric sleeve takes upwards of 30% more coating to fill-in the weave of the fabric and smooth-out its texture to achieve comparable performance.

In an effort to save money, some physicians have tried to re-sterilize the funnels. This has not presented a significant problem with the current KELLER FUNNEL made from nylon fabric as the coating does not hold up to the high temperatures and humidity of the autoclave cycle. It simply does not perform adequately after attempts to re-sterilize.

A plastic film with adhered HA lubricant is more durable. Thus, it provokes attempts to employ autoclave sterilization and reuse the sleeve.

However, single-use medical devices are desirable because they are generally regarded as presenting improved sterility upon retrieval from sterile packaging in a surgical field as compared to autoclaved parts transported around a hospital. Especially in the case where a tool is to be in intimate contact with an implant, and under circumstances (noted above) where there is a prevailing view that contamination is the cause of long-term complication, enforcing a single-use regimen for the delivery device is advisable for the sake of patient safety.

Indeed, the rapid commercial adoption of the KELLER FUNNEL (estimated to be used in more than 15% of silicone gel procedures in the United States as of the filing date hereof), is directly related to the many benefits imparted by the sleeve as compared to the traditional technique of silicone gel placement using primarily manual manipulation (i.e., insertion by squeezing and pushing the implant with the user's fingers and without a KELLER FUNNEL). In a market research study based on 241 survey respondents, physicians self-reported a 91% product satisfaction with the KELLER FUNNEL and 94% reported the “No-Touch” (primarily manual manipulation) technique to be an important benefit. Wiser, M., (November 2010) Funnel Opinion Panel Survey, WISERInsights, Miami, Fla. Contracture around breast implants is the most common medium- to long-term complication following breast augmentation. There is now clear evidence that bacteria can attach to the surface of breast implants at the time of surgery to form a biological coating (biofilm), which can go on to cause inflammation and contracture. Deva, A., Plastic Surgeons reveal reality of global hot topics in cosmetic surgery, Cosmetic Surgery Times, Publish Date: Aug. 1, 2011 by Lisette Hilton. Significant cost reductions to the health care industry in addition to reduced patient discomfort and lost productivity days would certainly be anticipated due to the implementation of techniques to reduce the introduction of autologous skin and parenchyma based flora at the time of surgical placement. It has been shown that use of the KELLER FUNNEL, and its associated “No-Touch” technique, reduces skin contact and potential contamination by 27 fold (p=0.00059) in a cadaver model. Moyer, et al, Sterility in Breast Implant Placement: The [KELLER FUNNEL] and the No Touch Technique, SESPRS 54th Annual Scientific Meeting, Naples, Fla. June 2011.

Features optionally applied to the film-based delivery device described above are advantageous in this regard. Specifically, the delivery systems are constructed from or incorporating heat shrink material. If subjected to autoclave temperatures (e.g., typically about 120° celsius (C.) at high steam pressure) the heat shrink material will cause the delivery device shape to change, making re-use physically impossible. In effect, autoclaving destroys the shape-wise utility of the product. It is noted that International Organization for Standardization (ISO) 17665 is an applicable industry standard that describes suitable autoclave conditions. Thus, it provides a safeguard to enforce the single-use (or disposable) model within the operating room.

By “heat shrink” what is meant is a polymer processed (or processable) so that it contracts or “shrinks” to a new configuration of reduced surface area upon reaching a threshold temperature. The material may be originally sourced in tubes/sleeves, sheets, tapes, and the like. Generally, heat-shrink material is manufactured from a thermoplastic material. Depending on the material employed, there are generally two ways that heat shrink may work. If the material contains many monomers, then when the tubing (for example) is heated the monomers polymerise. This increases the density of the material as the monomers become bonded together, therefore taking up less space. Accordingly, the volume of the material shrinks Heat shrink can also be expansion-based. This process involves producing the tubing (again for example) as normal, heating it to just above the polymer crystalline melting point and mechanically stretching the tubing (possibly by inflating it with a gas); finally, it is rapidly cooled. Later, when heated, the tubing will relax back to the un-expanded size. The material is often cross-linked through the use of electron beams, peroxides, or moisture. This cross-linking helps to make the tubing maintain its shape, both before and after shrinking

Where PET heat shrink is used in construction, the sleeve may start to become misshapen at about 82° C./180° F. When PVC heat shrink is used, the sleeve may start to become misshapen at about 100° C./212° F. Of course, other suitable heat shrink polymers can be employed.

In one embodiment, a longitudinally-contracting heat shrink tape is incorporated in a seam along the length of the funnel. The higher the shrink ratio (e.g., 4:1 vs. 2:1) along the length of the tape, the more deformation of the gross shape of the funnel body that will occur if subjected to the heat of the autoclave cycle. Regardless, any significant “crumpling” is beneficial in effectively scrapping the product to thwart reuse.

In another embodiment, the entire funnel is made of heat shrink material. In this case, the shrink material starts as an expanded, generally cylindrical sleeve that is heat-shaped over a generally conical mandrel (e.g., in the form of a stainless steel shell) during manufacture to define the funnel shape. The shaped delivery sleeve may be subsequently coated. When subjected to autoclave temperatures, the entire body shrinks into (roughly) a reduced-diameter, generally cylindrical sleeve so that it is no longer suitable for use as a delivery funnel as variously shown and described.

Certainly, medical devices are often constructed using heat shrink materials. It is common in catheter construction. However, the devices, systems, and methods described herein are distinguishable by way of the state of the material in the finished goods. Namely, those goods include heat shrink elements that remain enlarged or expanded (or at least partially expanded). Such expanded portions are unconstrained (or only partially constrained) in the final product so as to permit their contraction upon subsequent autoclave sterilization, thereby changing the bulk/gross dimensions and shape of the product so that it is no longer suitable for its original intended use.

Yet another embodiment concerns a multi-component funnel system. It may or may not include the autoclave-shrinkable elements as in the variations above.

The multi-component funnel system includes an inner and outer sleeve. The outer sleeve may be a tapered body as described above with the associated advantages of the smaller end of the funnel being suitable for receipt within an incision and the larger end better for receiving an implant. The inner sleeve may be a generally cylindrical sleeve or a second funnel-shaped body or element.

In either case, the inner sleeve can be shorter in length than the outer sleeve so that it basically only covers the implant. The inner sleeve is capable of receiving an implant that is highly textured, has a high surface friction, or is otherwise “grippy” (such as the newest generation of polyurethane (PU) covered silicone filled breast implants).

The exterior of the inner sleeve shields or masks an implant so that it can be easily squeezed through the funnel when set in a (relatively) small incision. The implant is delivered together with the inner sleeve into the breast pocket. After delivery, without the pressure of the outer sleeve bearing down upon the inner sleeve (alternatively referred to as an “implant jacket”), the implant jacket can be removed easily from the surgical pocket.

Certain other features are useful. One optional feature is a coating adhered to the exterior of the inner sleeve. Such coating ensures preferential slip between the sleeves (as opposed to slip between the implant and interior of the inner sleeve). Coating may or may not be used on the inner surface of the outer sleeve or funnel. Or the coating may be on the inner surface of the outer sleeve, with none on the inner sleeve.

It is generally preferred that at least one of the two sleeve surfaces contacting the other has a coating adhered thereto. Still, neither sleeve is necessarily coated in advance. A lubricant such as KY jelly could instead be employed to lubricate the sleeve-to-sleeve interface.

If one of the sleeve members is not coated, it may be preferred to leave the inner sleeve bare. As such, no coating-bearing member will be introduced into the surgical pocket during implant delivery.

Another optional feature is to employ a “reverse” taper to the inner sleeve (i.e., relative to the outer sleeve). In other words, the small end of the inner sleeve shape, which is typically oriented distally, will instead be oriented proximally. Such a shape (like draft angle on a molded part) assists in removal of this piece from around the implant once both items are introduced into the surgical pocket. Another approach to facilitate removal is to include extensions, loops or tabs on the inner sleeve that are easily gripped when the exterior sleeve is to be withdrawn from the surgical pocket.

When the heat shrink feature(s) are included, either one or both of the inner and outer sleeves are optionally constructed as discussed above or otherwise. One advantageous configuration utilizes only an inner sleeve of heat shrink tubing left in its original expanded configuration (e.g., as a generally cylindrical sleeve). Such a construction requires no additional shaping during production. Yet, to ensure that no component of the system is reused after autoclaving, it should be appreciated that only the outer sleeve need incorporate the heat shrink. A system so-configured avoids reuse of the outer sub-component of the system for delivering another type of implant (e.g., a non-textured silicone implant) which does not benefit from the use of the inner sleeve.

Each feature and/or embodiment of the devices, systems, and methods described herein can be used with any combination of other features and/or embodiments described, unless explicitly or implicitly noted otherwise. In this regard, the use of different reference numerals for similar elements of different embodiments should not be interpreted as meaning that those embodiments are not combinable. Such combinations may possess a variety of advantages. Still further, the scope of the present disclosure includes methods associated with and/or activities implicit to the use of the devices described. Acts of preparing the devices or systems described herein prior to use in a medical procedure are included within the scope of this disclosure.

### BRIEF DESCRIPTION OF THE DRAWINGS

The figures provided herein are not necessarily drawn to scale, with some components and features possibly being exaggerated for clarity. Each of the figures diagrammatically illustrates aspects of the invention. Of these:

FIGS. 1A-1C illustrate features of a system for packaging an implant in combination with a delivery sleeve according to one aspect of the invention;

FIGS. 2A and 2B show a delivery sleeve as it may be constructed and the effect heating for attempted sterilization and reuse, respectively;

FIGS. 3A-3D are construction views of another inventive delivery sleeve;

FIGS. 4A-4C illustrates sleeve components according to yet another variation.

Variation from the embodiments pictured is, of course, contemplated. Moreover, details commonly understood by those with skill in the art may be omitted from the figures.

### DETAILED DESCRIPTION

Various example embodiments are described herein. Reference is made to these examples in a non-limiting sense. They are provided to illustrate more broadly applicable aspects of the devices, systems, and methods described herein. Various changes may be made to these embodiments and equivalents may be substituted without departing from the scope of the disclosure. In addition, many modifications may be made to adapt a particular situation, material, composition of matter, process, process act(s) or step(s) to conform to the described or implied objective(s). All such modifications are intended to be within the scope of the claims made herein.

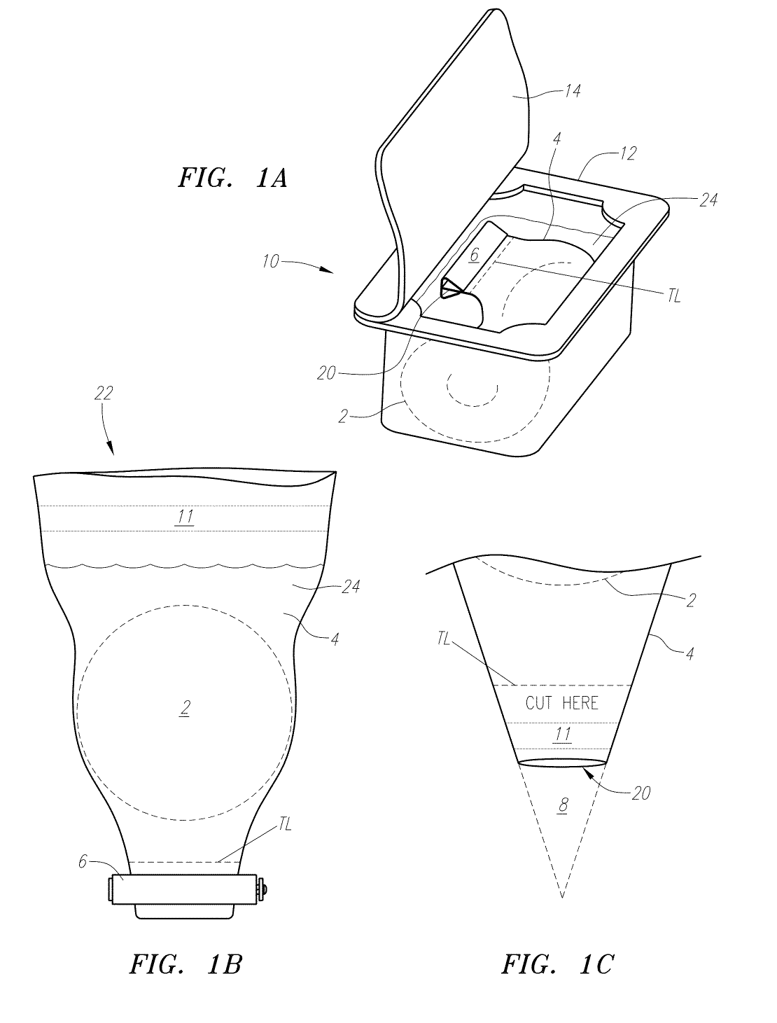

FIG. 1A illustrates an implant 2 set within a delivery sleeve 4, all within packaging 10 (container 12 with TYVEK cover 14 opened for the purpose of illustration). A clip 6 can be placed at the end of the sleeve 4 to close it off (alternative clip embodiments are shown in FIGS. 1A and 1B.)

Another option is to extend the sleeve 4 to a closed conical end 8 (as indicated by dashed line in FIG. 1C). In any case, a trim line “TL” may be provided toward the end of the sleeve 4 that has a location to coordinate with the size of the implant. Still further, instead of using a clip in FIG. 1C, tape or adhesive 11 (indicated as optional by a dotted band) may be used to seal the funnel across an already truncated distal end 20.

Tape or adhesive 11 may be used to similarly close and seal the proximal end 22 of the sleeve 4. In which case, the sleeve 4 may contain hydration fluid 24 (shown in FIG. 1B) for a lubricous hydrophilic coating. If not sealed within the sleeve 4, fluid 24 may be present within the sealed packaging 10 for the same purpose. In either case, adding fluid from an external source is avoided. Moreover, all implant and sleeve preparation can be performed in containment (i.e., inside the confines of the sterile tray).

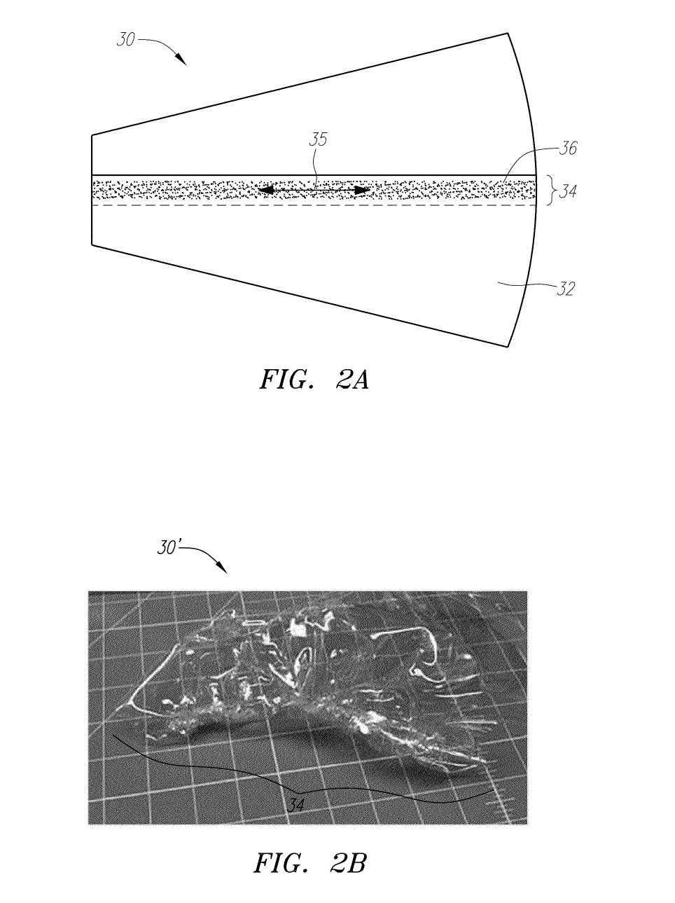

FIG. 2A is a construction view of another embodiment of sleeve 30. Here sleeve 4 comprises film 32 joined along a seam 34. Seam 34 incorporates one or more lengths of heat shrink material 36, oriented to contract along an axis as indicated by the double-arrow 35. An example material is 70% shrink PET at 0.0028″ thickness (as available from Dunstone, Inc.) cut in strips (or tape) made from rings of sleeve material. In other words, the strips are provided so as to shrink along longitudinal axis 35.

In assembly, the layered structure is set within a “scissors” type heat sealer (using an thermal insulating batten within the funnel to protect both sides of the film from being welded together) to weld one or more pieces of heat shrink tubing along an overlapped or “lap” joint seam between length-wise ends of the film material from which the sleeve 4 is constructed. In this example, film 32 comprises 0.008″ thick PVC (as available from Adam's Plastic of Chicago, Ill.).

In any case, when constrained during heating, contraction of the heat shrink is prevented but the elements are bonded together. In another construction approach, heat shrink material can be sandwiched between opposing layers of double-sided tape.

In any such device, autoclaving results in a “crumpled” and unusable device 30′ as illustrated in FIG. 2B. It becomes so-shaped because of contraction along the lap joint seam 34.

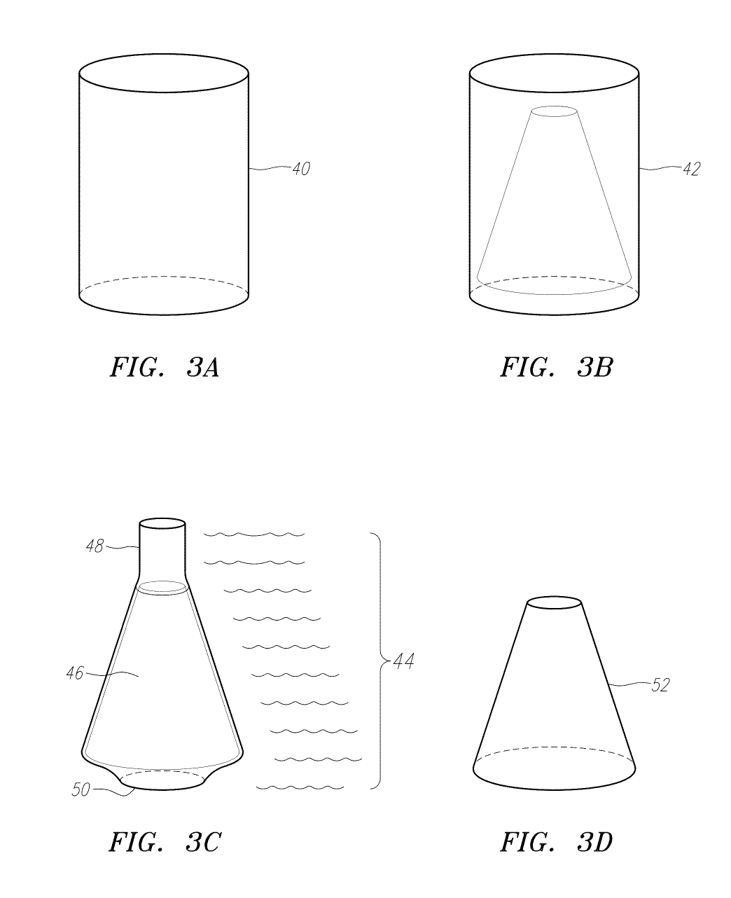

FIGS. 3A-3D illustrate another manufacturing approach to a heat shrink (and further shrinkable) delivery device sleeve. FIG. 3A shows a tube 40 of heat shrink material per above. The tube can include a seam (not shown) or be seamless.

In any case, as provided by a supplier, the material is generally cylindrical or provided in a “lay flat” configuration. In FIG. 3B, it is set over a conical mandrel 42 and subjected to heating (e.g., by hot air 44 from a heatgun, within an oven, or otherwise.)

In FIG. 3C, a funnel preform 46 is shown as shaped upon the mandrel 42. After ends 48, 50 are trimmed off (or otherwise removed), the shaping of the delivery device is complete. In FIG. 30, delivery sleeve 52 is shown ready for coating by conventional techniques, if a coating is to be applied to the final product.

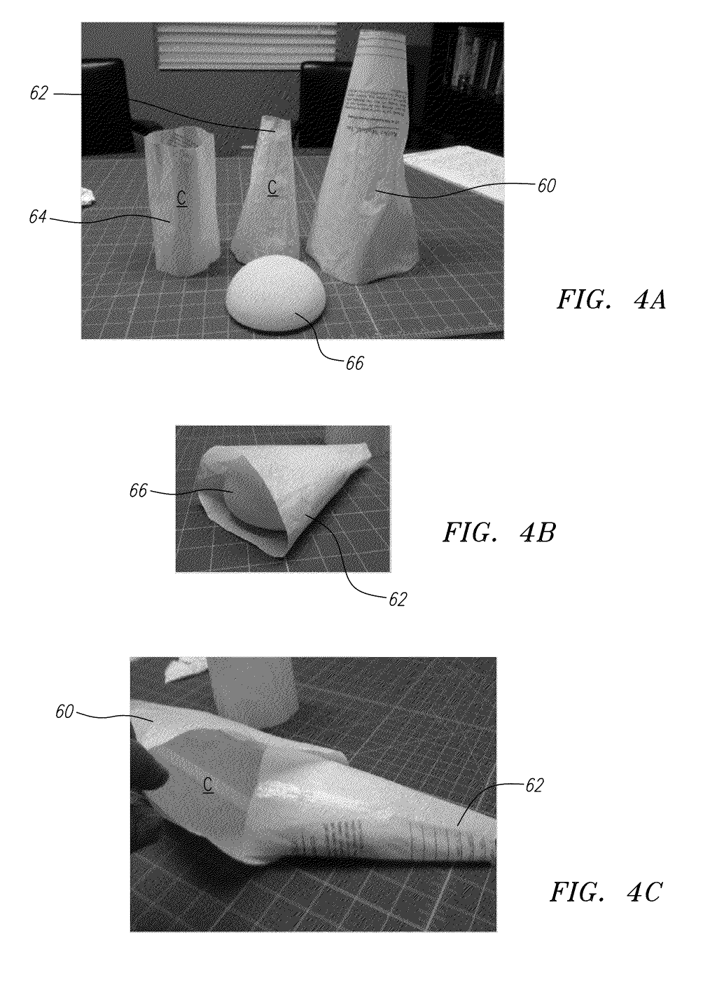

FIG. 4A illustrates sleeve components according to yet another embodiment. These may be produced in accordance with the above or fabricated otherwise. The set of components include an outer sleeve 60 and alternative inner sleeves 62, 64 (tapered and cylindrical, respectively). Lubricious coating may be adhered to the items, variously.

In one example, the exterior of the inner sleeve and interior of the outer sleeve are provided with hydrophilic coating “C” as indicated. However, no coating is necessary, or might instead be user-applied.

FIG. 4B shows an implant 66 received within inner sleeve 62 so-serving as an implant jacket. In FIG. 4C, the implant 66 and jacket 62 (alternatively, jacket 64) are received within the outer sleeve 60. As illustrated, the tapers of the sleeve 60 and 62 are set opposite one another for reasons described above with respect to implant delivery. In other words, from its proximal end to its distal end, jacket 62 tapers inwardly, or narrows. The taper on outer sleeve 60 is reversed, such that sleeve 60 tapers outwardly, or broadens, from its proximal end to its distal end.

While embodiments having a hydrophilic coating have been described, it should be understood that hydrophilic coatings are but one example of lubricious coatings that can be used. Generally, the surface of the sleeve that will be in contact with the implant is lubricated. The sleeve can be packaged with the lubricant already in place on the implant-contacting surface or the lubricant can be applied by the medical professional after removal from the packaging. The lubricant can also be applied to the implant itself in addition to, or instead of, being present on the sleeve surface. A number of lubricants can be used, not limited to hydrophilic coatings.

The scope of the present disclosure includes these methods and others set forth above in terms of method of manufacture, preparation and/or use. The methods may be performed using the subject devices and sometimes by other means.

The methods may include the act of providing a suitable device. Such provision may be performed by the end user. In other words, the act of “providing” merely requires that the end user access, approach, position, set-up, grasp or otherwise obtain the requisite device for the subject method. Methods recited herein may be carried out in any order of the recited events which is logically possible, as well as in the recited order of events.

Exemplary embodiments, together with details regarding material selection and manufacture have been set forth above. As for other details of the devices, systems, and methods described herein, these may be appreciated in connection with the above-referenced patents and publications as well as in connection with the general knowledge of those with skill in the art. The same may hold true with respect to method-based embodiments in terms of additional acts as commonly or logically employed.

In addition, though the devices, systems, and methods described herein have been done in reference to several examples, optionally incorporating various features, the devices, systems, and methods described herein are not to be limited to that which is described or indicated as contemplated with respect to each variation. Changes may be made to the embodiments described and equivalents (whether recited herein or not included for the sake of brevity) may be substituted without departing from the true spirit and scope of the present disclosure. In addition, where a range of values is provided explicitly or implicitly, it is understood that every intervening value, between the upper and lower limit of that range and any other stated or intervening value in that stated range is encompassed.

Also, it is contemplated that any optional feature of the embodiments described may be set forth and claimed independently, or in combination with any one or more of the features described herein. Stated otherwise, it is to be understood that each of the embodiments described herein independently offer a valuable contribution to the state of the art. So too do the various other possible combinations of the improvements/features described herein and/or incorporated by reference, any of which may be claimed.

1. An apparatus for inserting a pre-filled or silicone prosthesis into a surgical pocket, the apparatus comprising:
a tapered sleeve, open at a distal end for use, the sleeve comprising expanded heat shrink material adapted to contract upon exposure to autoclave conditions.

2. The apparatus of claim 1, wherein the sleeve includes a seam incorporating heat shrink material.

3. The apparatus of claim 1, wherein the sleeve consists essentially of the heat shrink material.

4. The apparatus of claim 1, wherein the sleeve comprises lubricant.

5. The apparatus of claim 1, wherein the sleeve is a first sleeve and the apparatus further comprises a second sleeve open at a distal end for use, one sleeve being received within the other.

6. The apparatus of claim 5, wherein at least one of the sleeves comprises a dry hydrophilic coating adhered along an interface between the sleeves.

7. The apparatus of claim 5, wherein the second sleeve is received within the first sleeve, the second sleeve being adapted to receive an implant.

8. The apparatus of claim 5, wherein the first sleeve is received within the second sleeve, the first sleeve being adapted to receive an implant.

9. The apparatus of claim 5, wherein the second sleeve is tapered.

10. The apparatus of claim 9, wherein the first and second sleeves are tapered in opposite directions.

11. The apparatus of claim 10, wherein both sleeves have an open proximal end.

12. A system for inserting a pre-filled or silicone prosthesis into a surgical pocket, the system comprising:
a flexible tapered sleeve open at a larger end and closed at a smaller end, and a prefilled prosthesis received within the sleeve in packaged combination, the sleeve adapted to deliver the implant only upon opening the closed end.

13. The system of claim 12, where the system includes a clip closing the sleeve, the clip adapted for removal to open the sleeve.

14. The system of claim 12, wherein the closed end is at a terminal portion of the sleeve, the terminal portion to be cut off to open the sleeve.

15. A system for inserting a pre-filled or silicone prosthesis into a surgical pocket, the system comprising:
an inner sleeve and an outer sleeve, the inner sleeve adapted to be received within the outer sleeve, both sleeves having an open distal end for use, the outer sleeve being tapered inwardly from a proximal end to the distal end and the inner sleeve being tapered outwardly from a proximal end to the distal end.
